(19) United States
(12) Patent Application Publication
(10) Pub. No.: US 2017/0079675 A1
(43) Pub. Date: Mar. 23, 2017

(54) TAPERED MEMBRANE REMOVING FORCEPS

(71) Applicant: Katalyst Surgical, LLC, Chesterfield, MO (US)

(72) Inventors: Gregg D. Scheller, Wildwood, MO (US); Eric J. Bass, Webster Groves, MO (US)

(73) Assignee: Katalyst Surgical, LLC, Chesterfield, MO (US)

(21) Appl. No.: 15/252,289

(22) Filed: Aug. 31, 2016

Related U.S. Application Data
(60) Provisional application No. 62/220,553, filed on Sep. 18, 2015.

(51) Int. Cl. A61B 17/30; A61B 17/00

(52) U.S. Cl. CPC A61B 17/30; A61B 17/00234; A61B 2017/305; A61B 2017/0046

(57) ABSTRACT

A tapered membrane removing forceps may include a first tapered forceps jaw and a second tapered forceps jaw. Each tapered forceps jaw may include a forceps jaw distal end, a forceps jaw proximal end, a superior face, and a medial face. Each superior face may include a tapered portion having a tapered angle. Each tapered angle may be an angle in a range of 19.0 to 23.0 degrees. Each tapered forceps jaw may be at least partially disposed in a hypodermic tube wherein the tapered forceps jaw distal ends extend from a distal end of the hypodermic tube. A compression of an actuation structure may be configured to extend the hypodermic tube relative to each tapered forceps jaw.

### CROSS-REFERENCE TO RELATED APPLICATIONS

This Application claims the benefit of U.S. Provisional Application No. 62/220,553, filed Sep. 18, 2015.

### FIELD OF THE INVENTION

The present disclosure relates to a surgical instrument, and, more particularly, to a microsurgical forceps.

### BACKGROUND OF THE INVENTION

A microsurgical forceps may be used to perform a microsurgical procedure, e.g., an ophthalmic surgical procedure. For example, a surgeon may use a forceps to grasp and manipulate tissues or other surgical instruments to perform portions of a surgical procedure. A particular microsurgical procedure may require a surgeon to separate a first tissue from a second tissue without causing trauma to at least one of the tissues. Such a separation procedure may be particularly difficult for a surgeon to perform if the tissue surface geometry is not flat, e.g., if the tissue surface geometry is convex. For example, an ophthalmic surgeon may be required to remove an internal limiting membrane from a patient's retina without causing trauma to the patient's retina. Accordingly, there is a need for a microsurgical forceps that enables a surgeon to separate a first tissue from a second tissue without causing significant trauma to at least one of the tissues. One method of reducing trauma to a retina requires complete visualization of the retina which allows a surgeon to estimate a depth of penetration of a forceps. Accordingly, there is a need for a microsurgical forceps that increases a surgeon's ability to visualize a retina during a surgical procedure.

### BRIEF SUMMARY OF THE INVENTION

The present disclosure provides a tapered membrane removing forceps. In one or more embodiments, a tapered membrane removing forceps may comprise a first tapered forceps jaw and a second tapered forceps jaw. Illustratively, each tapered forceps jaw may comprise a forceps jaw distal end, a forceps jaw proximal end, a superior face, and a medial face. In one or more embodiments, each superior face may comprise a tapered portion having a tapered angle. Illustratively, each tapered angle may comprise an angle in a range of 19.0 to 23.0 degrees. In one or more embodiments, each tapered forceps jaw may be at least partially disposed in a hypodermic tube wherein the tapered forceps jaw distal ends extend from a distal end of the hypodermic tube. Illustratively, a compression of an actuation structure may be configured to extend the hypodermic tube relative to each tapered forceps jaw.

### BRIEF DESCRIPTION OF THE DRAWINGS

The above and further advantages of the present invention may be better understood by referring to the following description in conjunction with the accompanying drawings in which like reference numerals indicate identical or functionally similar elements:

FIGS. 1A, 1B, 1C, 1D, 1E, 1F, and 1G are schematic diagrams illustrating a tapered membrane removing forceps tip;

FIG. 2A is a schematic diagram illustrating an exploded view of a tapered membrane removing forceps assembly;

FIGS. 2B and 2C are schematic diagrams illustrating an assembled tapered membrane removing forceps;

FIGS. 3A, 3B, and 3C are schematic diagrams illustrating a gradual closing of a tapered membrane removing forceps tip;

FIGS. 4A, 4B, 4C, and 4D are schematic diagrams illustrating a surgical procedure.

### DETAILED DESCRIPTION OF AN ILLUSTRATIVE EMBODIMENT

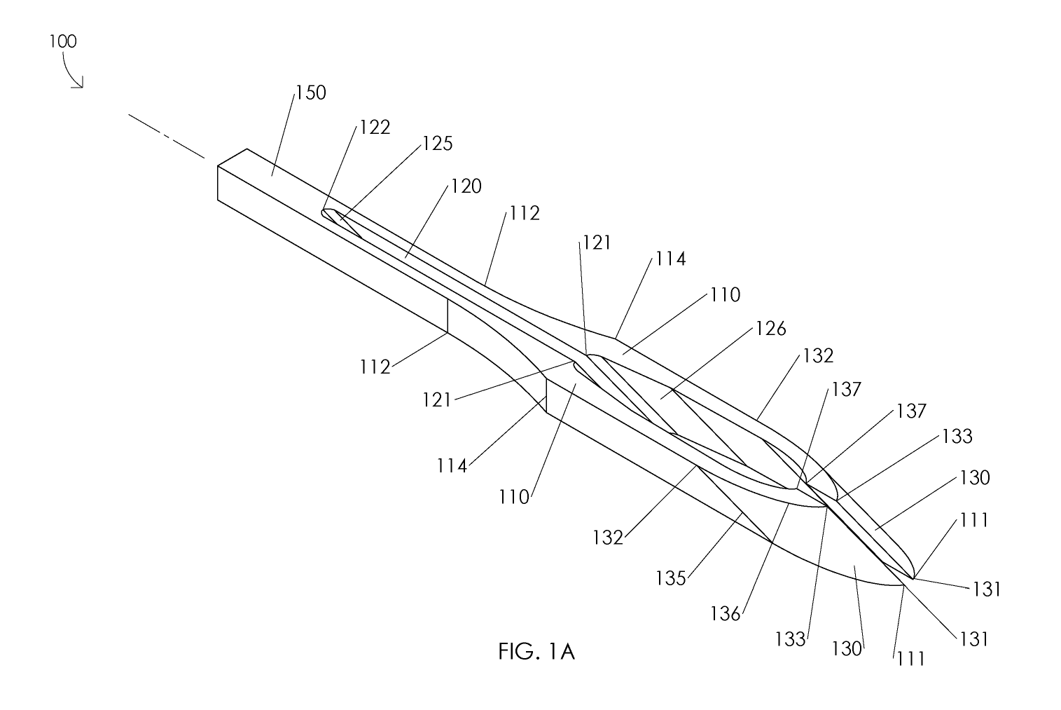

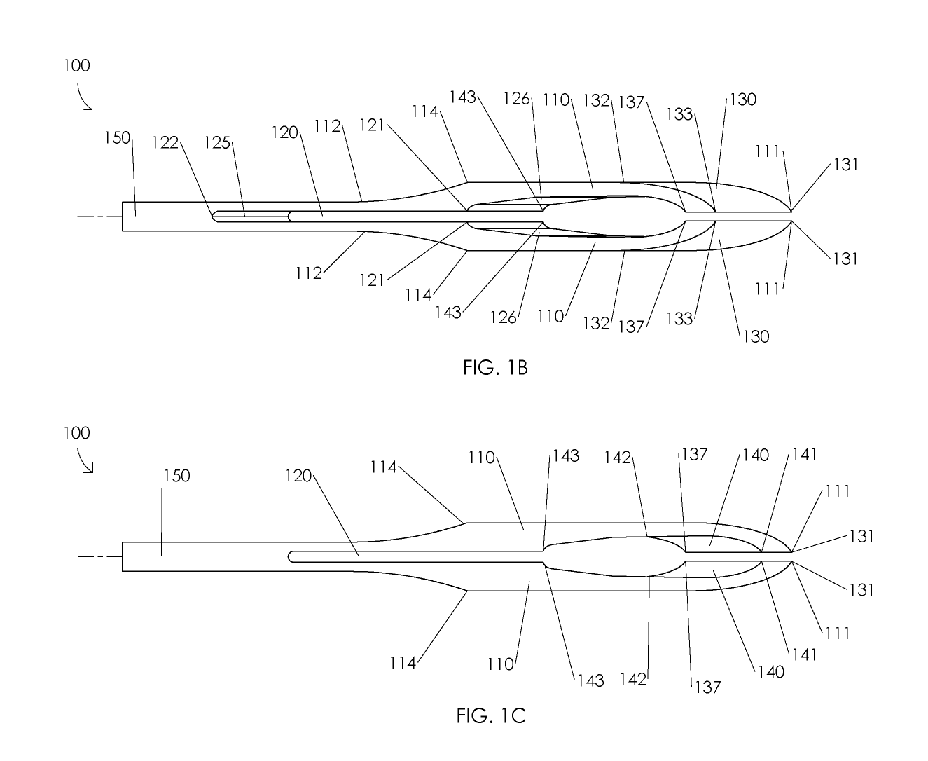

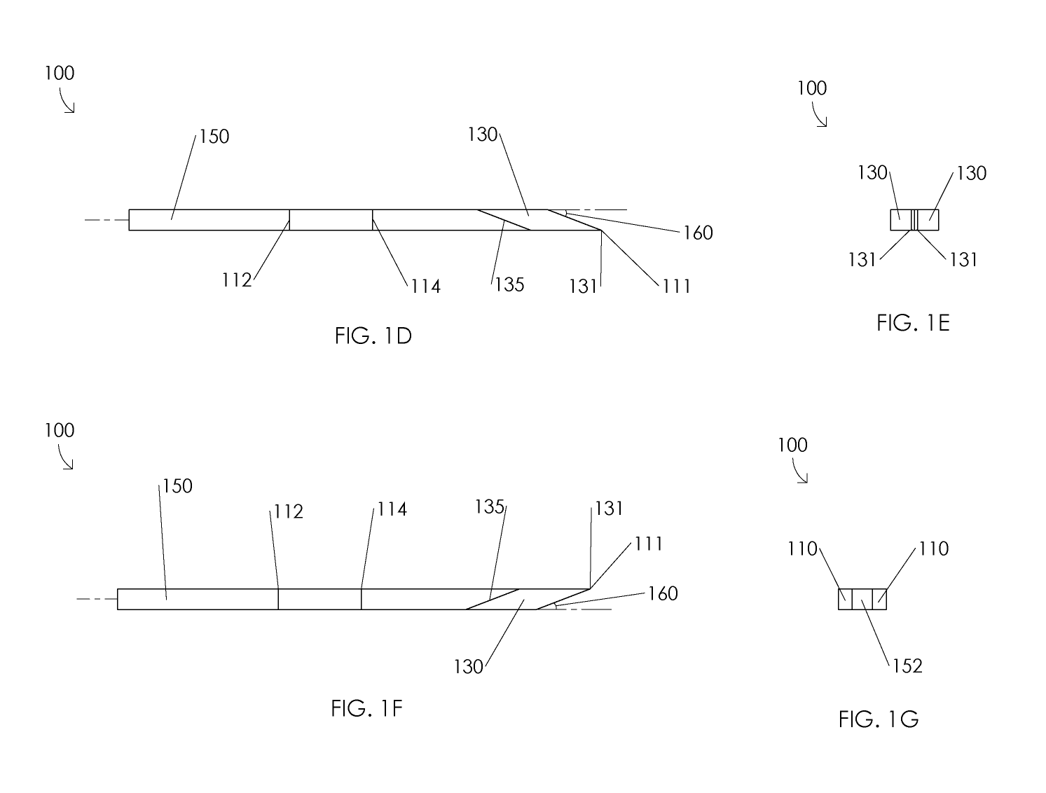

FIGS. 1A, 1B, 1C, 1D, 1E, 1F, and 1G are schematic diagrams illustrating a tapered membrane removing forceps tip 100. FIG. 1A illustrates an isometric view of a tapered membrane removing forceps tip 100. In one or more embodiments, a tapered membrane removing forceps tip 100 may comprise a first tapered forceps jaw 110 and a second tapered forceps jaw 110. Illustratively, each tapered forceps jaw 110 may comprise a tapered forceps jaw distal end 111, a tapered forceps jaw proximal end 112, a lateral projection 114, a medial face 126, a superior face 130, a superior distal shoulder 137, and a superior proximal shoulder 121. In one or more embodiments, each superior face 130 may comprise a superior face distal end 131, a superior face proximal end 132, a superior face superior end 133, a superior face lateral interface 135, and a superior face superior interface 136. Illustratively, each lateral projection 114 may be disposed between tapered forceps jaw proximal end 112 and tapered forceps jaw distal end 111, e.g., each lateral projection 114 may be disposed between tapered forceps jaw proximal end 112 and superior face lateral interface 135. In one or more embodiments, each superior face 130 may be disposed on a distal portion of tapered forceps jaw 110 wherein superior face distal end 131 is tapered forceps jaw distal end 111. Illustratively, a first tapered forceps jaw 110 may be separated from a second tapered forceps jaw 110 by a tapered aperture 120. In one or more embodiments, tapered aperture 120 may be disposed between each medial face 126. Illustratively, tapered aperture 120 may comprise a tapered aperture proximal end 122 and a tapered aperture tapered portion 125. In one or more embodiments, tapered aperture tapered portion 125 may be disposed between tapered aperture proximal end 122 and tapered forceps jaw proximal end 112. Illustratively, tapered aperture tapered portion 125 may be tapered at any angle less than 90.0 degrees relative to a line tangent to a superior portion of tapered membrane removing forceps tip 100. In one or more embodiments, tapered aperture tapered portion 125 may be tapered at an angle in a range of 19.0 to 23.0 degrees relative to a line tangent to a superior portion of tapered membrane removing forceps tip 100, e.g., tapered aperture tapered portion 125 may be tapered at an angle of 21.0 degrees relative to a line tangent to a superior portion of tapered membrane removing forceps tip 100. Illustratively, tapered aperture tapered portion 125 may be tapered at an angle of less than 19.0 degrees or greater than 23.0 degrees relative to a line tangent to a superior portion of tapered membrane removing forceps tip 100. For example, tapered aperture tapered portion 125 may be tapered at an angle relative to a line tangent to a superior portion of tapered membrane removing forceps tip 100 configured to match an angle of approach during a surgical procedure. In one or more embodiments, tapered membrane removing forceps tip 100 may be manufactured from any suitable material, e.g., polymers, metals, metal alloys, etc., or from any combination of suitable materials.

FIG. 1B illustrates a top view of a tapered membrane removing forceps tip 100. Illustratively, each tapered forceps jaw 110 may comprise an inferior proximal shoulder 143. In one or more embodiments, each inferior proximal shoulder 143 may be disposed between a medial face 126 of a first tapered forceps jaw 110 and a medial face 126 of a second tapered forceps jaw 110. Illustratively, inferior proximal shoulder 143 may be disposed between superior proximal shoulder 121 and superior distal shoulder 137. FIG. 1C illustrates a bottom view of a tapered membrane removing forceps tip 100. Illustratively, each tapered forceps jaw 110 may comprise an inferior face 140 having an inferior face distal end 141 and an inferior face proximal end 142. In one or more embodiments, inferior face distal end 141 may be disposed between tapered forceps jaw distal end 111 and superior distal shoulder 137. Illustratively, inferior face proximal end 142 may be disposed between superior distal shoulder 137 and inferior proximal shoulder 143.

FIG. 1D illustrates a side view of a tapered membrane removing forceps tip 100. FIG. 1E illustrates a front view of a tapered membrane removing forceps tip 100. FIG. 1F illustrates a side view of a tapered membrane removing forceps tip 100 rotated 180 degrees. FIG. 1G illustrates a back view of a tapered membrane removing forceps tip 100. Illustratively, tapered membrane removing forceps tip 100 may comprise a tapered angle 160. In one or more embodiments, tapered angle 160 may comprise an angle between a line tangent to a superior surface of tapered forceps jaw 110 and superior face 130. Illustratively, tapered angle 160 may comprise any angle less than 90.0 degrees. In one or more embodiments, tapered angle 160 may comprise an angle in a range of 19.0 to 23.0 degrees, e.g., tapered angle 160 may comprise an angle of 21.0 degrees. Illustratively, tapered angle 160 may comprise an angle of less than 19.0 degrees or greater than 23.0 degrees. In one or more embodiments, tapered angle 160 may be configured to match an angle of approach during a surgical procedure. For example, a surgeon may manipulate an orientation of a tapered membrane removing forceps tip 100 until no portion of superior face 130 is visible in the surgeon's line-of-sight.

Illustratively, tapered membrane removing forceps tip 100 may be manufactured with dimensions configured for performing microsurgical procedures, e.g., ophthalmic surgical procedures. In one or more embodiments, tapered membrane removing forceps tip 100 may be manufactured from a blank 150. In one or more embodiments, tapered membrane removing forceps tip 100 may be manufactured by modifying blank 150, e.g., with an electric discharge machine, a laser, a file, deep reactive ion etching, or any suitable modification means. Illustratively, tapered angle 160 may be formed by orienting blank 150 at tapered angle 160 relative to a wire of an electric discharge machine and actuating the wire through blank 150 at the tapered angle 160. In one or more embodiments, tapered membrane removing forceps tip 100 may be manufactured by a 3D printing process. For example, tapered membrane removing forceps tip 100 may be manufactured by selective laser sintering, selective heat sintering, selective laser melting, electron-beam melting, direct metal laser sintering, electron beam freeform fabrication, etc.

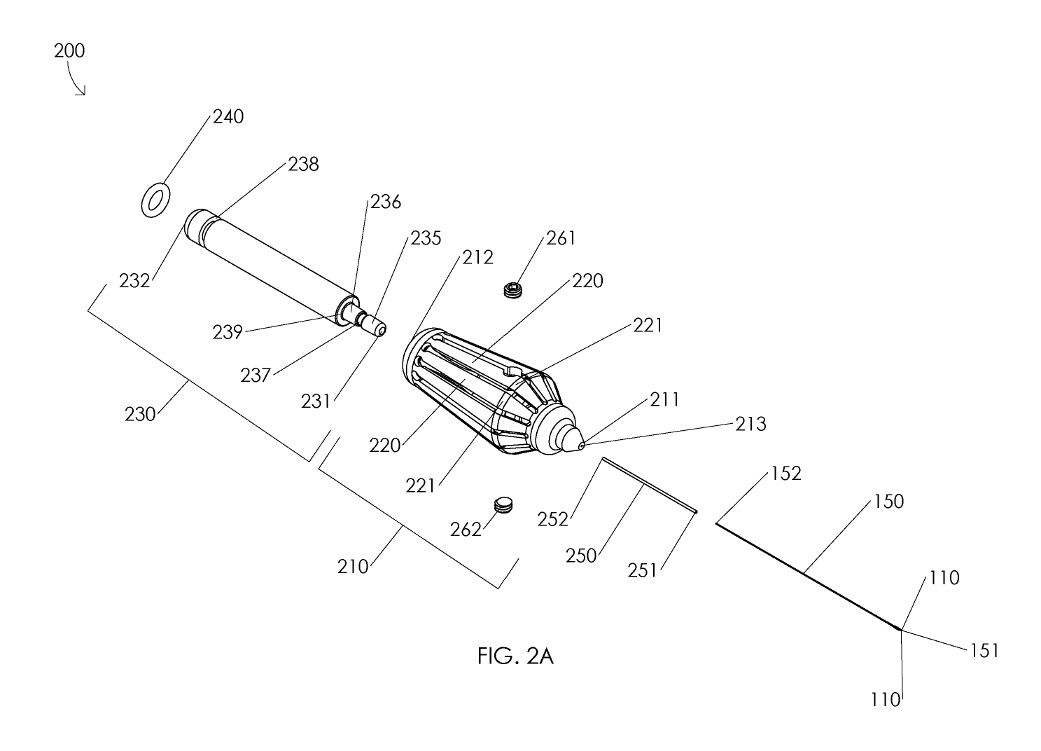

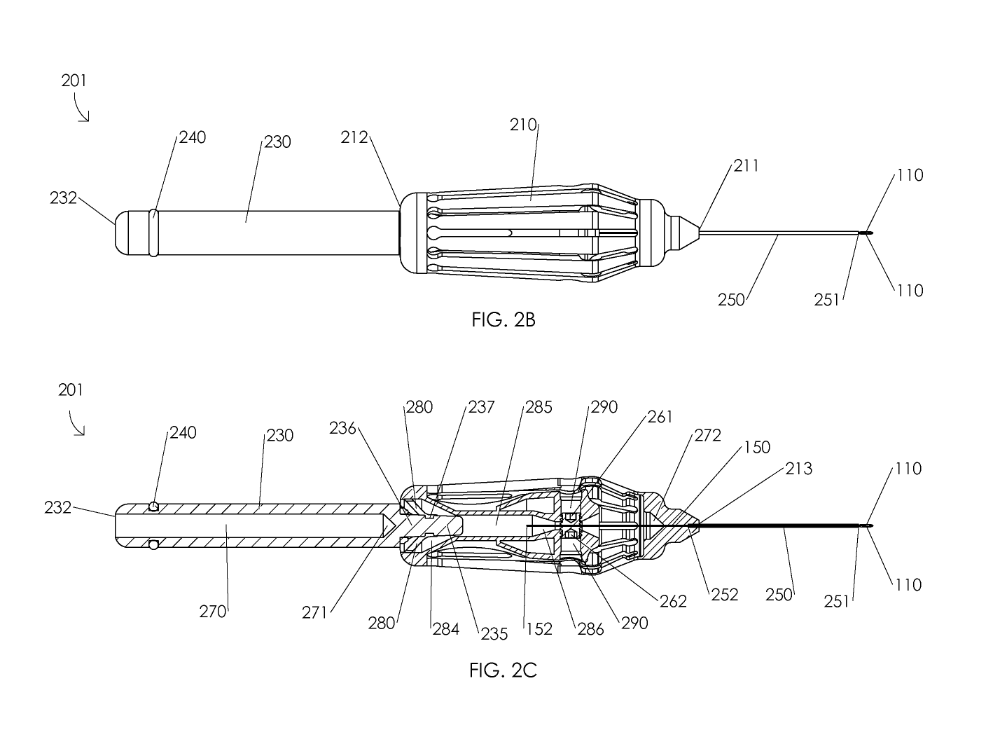

FIG. 2A is a schematic diagram illustrating an exploded view of a tapered membrane removing forceps assembly 200. Illustratively, a tapered membrane removing forceps assembly 200 may comprise an actuation structure 210, a removable handle 230, a gage indicator 240, a hypodermic tube 250, a blank 150, a superior setscrew 261, and an inferior setscrew 262. In one or more embodiments, an actuation structure 210 may comprise an actuation structure distal end 211, an actuation structure proximal end 212, a plurality of actuation arms 220, and a hypodermic tube housing 213. Illustratively, each actuation arm 220 of actuation structure 210 may comprise at least one extension mechanism 221. In one or more embodiments, removable handle 230 may comprise a removable handle distal end 231, a removable handle proximal end 232, a barb head 235, a barb base 236, a barb channel 237, a gage indicator housing 238, and an actuation structure interface 239. Illustratively, hypodermic tube 250 may comprise a hypodermic tube distal end 251 and a hypodermic tube proximal end 252. In one or more embodiments, blank 150 may comprise a blank distal end 151, a blank proximal end 152, and a tapered membrane removing forceps tip 100.

FIGS. 2B and 2C are schematic diagrams illustrating an assembled tapered membrane removing forceps 201. FIG. 2B illustrates a side view of an assembled tapered membrane removing forceps 201. FIG. 2C illustrates a cross-sectional view of an assembled tapered membrane removing forceps 201. Illustratively, gage indicator 240 may be disposed within gage indicator housing 238. In one or more embodiments, gage indicator 240 may be configured to visually indicate a size of hypodermic tube 250, e.g., gage indicator 240 may comprise a ring colored to visually indicate an outer diameter of hypodermic tube 250. Illustratively, removable handle 230 may comprise an inner bore 270 and an inner bore distal taper 271. In one or more embodiments, inner bore 270 may have an inner bore distal end and an inner bore proximal end wherein the inner bore proximal end is adjacent to the removable handle proximal end 232. Illustratively, inner bore distal taper 271 may be disposed between a distal end of inner bore 270 and barb base 236. In one or more embodiments, actuation structure 210 may comprise an inner nosecone 272, a plurality of fingers 280, an inner chamber proximal taper 284, an inner chamber 285, an inner chamber distal taper 286, and a setscrew housing 290. Illustratively, inner nosecone 272 may be disposed between setscrew housing 290 and actuation structure distal end 211. In one or more embodiments, setscrew housing 290 may be disposed between inner chamber distal taper 286 and inner nosecone 272. Illustratively, inner chamber distal taper 286 may be disposed between inner chamber 285 and setscrew housing 290. In one or more embodiments, inner chamber 285 may be disposed between inner chamber proximal taper 284 and inner chamber distal taper 286. Illustratively, each finger 280 of the plurality of fingers 280 may be disposed in inner chamber proximal taper 284.

Illustratively, a portion of removable handle 230 may be disposed within a portion of actuation structure 210, e.g., removable handle distal end 231 may be disposed within actuation structure 210. In one or more embodiments, barb head 235 may be disposed within actuation structure 210 wherein barb head 235 is disposed in inner chamber 285 and inner chamber proximal taper 284. Illustratively, barb base 236 may be disposed within actuation structure 210 wherein barb base 236 is disposed in inner chamber proximal taper 284. In one or more embodiments, barb channel 237 may be disposed within actuation structure 210 wherein barb channel 237 is disposed in inner chamber proximal taper 284. Illustratively, each finger 280 of the plurality of fingers 280 may be partially disposed in barb channel 237.

In one or more embodiments, a portion of removable handle 230 may be temporarily fixed within actuation structure 210, e.g., barb head 235, barb base 236, and barb channel 237 may be temporarily fixed within actuation structure 210. Illustratively, each finger 280 of the plurality of fingers 280 may be configured to temporarily fix a portion of removable handle 230 within actuation structure 210. In one or more embodiments, each finger 280 of the plurality of fingers 280 may be configured to temporarily fix a portion of removable handle 230 within actuation structure 210 by a snap fit, e.g., each finger 280 of the plurality of fingers 280 may be configured to temporarily fix a portion of removable handle 230 within actuation structure 210 by a torsional snap fit. Illustratively, a portion of removable handle 230 may be temporarily fixed within actuation structure 210 by a force of friction, e.g., a portion of removable handle 230 may be temporarily fixed within actuation structure 210 by an interference fit. In one or more embodiments, a portion of removable handle 230 may be disposed within a portion of actuation structure 210 wherein actuation structure interface 239 is adjacent to actuation structure proximal end 212.

Illustratively, a surgeon may optionally remove a portion of removable handle 230 from a portion of actuation structure 210. For example, a surgeon may optionally remove removable handle 230 from actuation structure 210 to grasp actuation structure wherein a portion of the surgeon's palm is adjacent to actuation structure proximal end 212. In one or more embodiments, a surgeon may optionally remove removable handle 230 from actuation structure 210 by pulling removable handle 230 out from inner chamber proximal taper 284. Illustratively, a surgeon may optionally insert removable handle 230 into actuation structure 210 by pushing removable handle 230 into inner chamber proximal taper 284. In one or more embodiments, a surgeon may perform a first portion of a surgical procedure with removable handle 230 disposed within actuation structure 210. Illustratively, the surgeon may perform a second portion of the surgical procedure with removable handle 230 removed from actuation structure 210. In one or more embodiments, the surgeon may perform a third portion of the surgical procedure with removable handle 230 disposed within actuation structure 210. Illustratively, the surgeon may perform a fourth portion of the surgical procedure with removable handle 230 removed from actuation structure 210.

In one or more embodiments, a portion of hypodermic tube 250 may be disposed in a portion of actuation structure 210, e.g., hypodermic tube proximal end 251 may be disposed in a portion of actuation structure 210. Illustratively, a portion of hypodermic tube 250 may be disposed in hypodermic tube housing 213, e.g., hypodermic tube proximal end 252 may be disposed in hypodermic tube housing 213. In one or more embodiments, a portion of hypodermic tube 250 may be fixed within a portion of actuation structure 210, e.g., a portion of hypodermic tube 250 may be fixed within a portion of actuation structure 210 by an adhesive, a weld, a force of friction, etc.

Illustratively, blank 150 may be disposed in hypodermic tube 250 and actuation structure 210, e.g., blank 150 may be disposed in hypodermic tube 250 an actuation structure 210 wherein blank proximal end 152 is disposed in actuation structure 210 and blank distal end 151 extends from hypodermic tube distal end 251. In one or more embodiments, blank 150 may be disposed in hypodermic tube 250, inner nosecone 272, setscrew housing 290, inner chamber distal taper 286, and inner chamber 285. Illustratively, superior setscrew 261 and inferior setscrew 262 may be disposed within setscrew housing 290. In one or more embodiments, blank 150 may be fixed in a position relative to actuation structure proximal end 212 and hypodermic tube 250, e.g., superior setscrew 261 and inferior setscrew 262 may be configured to fix blank 150 in a position relative to actuation structure proximal end 212 and hypodermic tube 250. Illustratively, a portion of blank 150 may be disposed between superior setscrew 261 and inferior setscrew 262 wherein the portion of blank 150 is fixed in a position relative to actuation structure proximal end 212 and hypodermic tube 250 by a force applied to the portion of blank 150 by superior setscrew 261 and inferior setscrew 262.

In one or more embodiments, a compression of actuation structure 210 may be configured to extend actuation structure distal end 211 relative to actuation structure proximal end 212. Illustratively, a compression of actuation structure 210 may be configured to extend hypodermic tube 250 relative to blank 150. In one or more embodiments, a compression of actuation structure 210 may be configured to extend hypodermic tube distal end 251 over a portion of first and second tapered forceps jaw 110, e.g., a compression of actuation structure 210 may be configured to extend hypodermic tube distal end 251 over first tapered forceps jaw proximal end 112 and over second tapered forceps jaw proximal end 112. Illustratively, a compression of actuation structure 210 may be configured to decrease a distance between first tapered forceps jaw medial face 126 and second tapered forceps jaw medial face 126. In one or more embodiments, a compression of actuation structure 210 may be configured to close tapered membrane removing forceps tip 100.

In one or more embodiments, a decompression of actuation structure 210 may be configured to retract actuation structure distal end 211 relative to actuation structure proximal end 212. Illustratively, a decompression of actuation structure 210 may be configured to retract hypodermic tube 250 relative to blank 150. In one or more embodiments, a decompression of actuation structure 210 may be configured to retract hypodermic tube distal end 251 away from a portion of first and second tapered forceps jaw 110, e.g., a decompression of actuation structure 210 may be configured to retract hypodermic tube distal end 251 away from first tapered forceps jaw proximal end 112 and away from second tapered forceps jaw proximal end 112. Illustratively, a decompression of actuation structure 210 may be configured to increase a distance between first tapered forceps jaw medial face 126 and second tapered forceps jaw medial face 126. In one or more embodiments, a decompression of actuation structure 210 may be configured to open tapered membrane removing forceps tip 100.

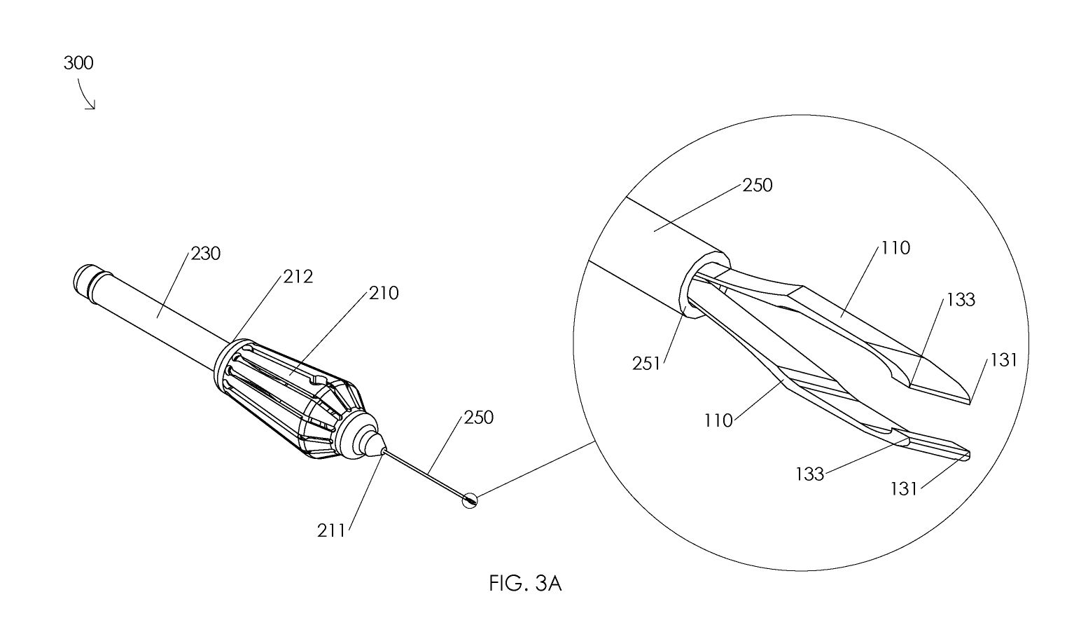

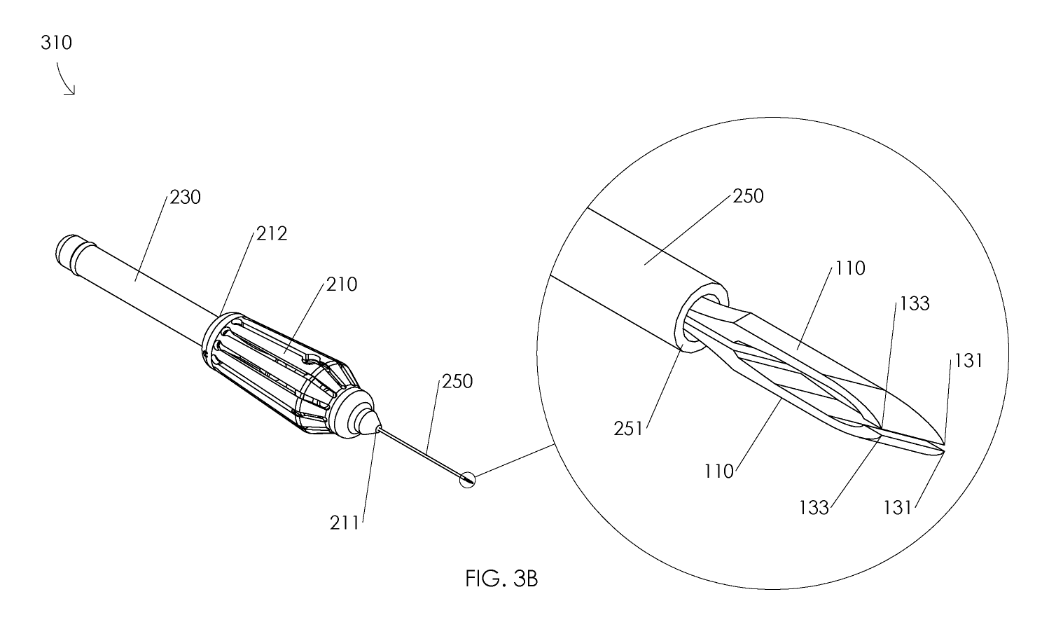

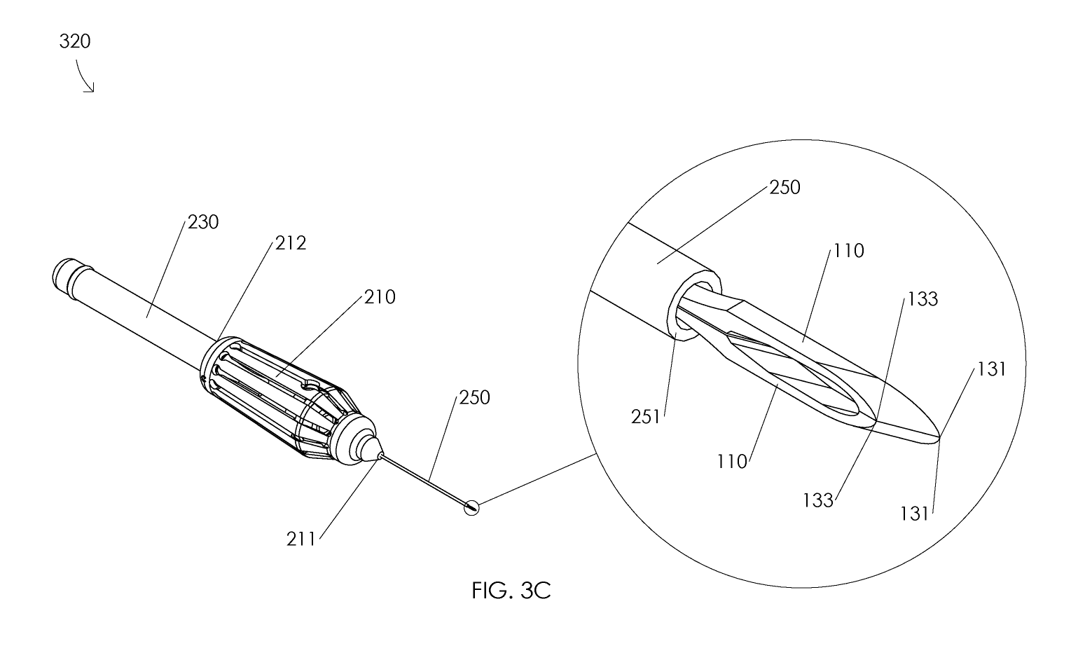

FIGS. 3A, 3B, and 3C are schematic diagrams illustrating a gradual closing of a tapered membrane removing forceps tip 100. FIG. 3A illustrates an isometric view of an open tapered membrane removing forceps 300. Illustratively, tapered membrane removing forceps tip 100 may comprise an open tapered membrane removing forceps 300 when actuation structure 210 is fully decompressed. In one or more embodiments, tapered membrane removing forceps tip 100 may comprise an open tapered membrane removing forceps 300 when first tapered forceps jaw 110 is fully separated from second tapered forceps jaw 110. Illustratively, first superior face distal end 131 may be separated from second superior face distal end 131 by a distance in a range of 0.002 to 0.004 inches when tapered membrane removing forceps tip 100 comprises an open tapered membrane removing forceps 300, e.g., first superior face distal end 131 may be separated from second superior face distal end 131 by a distance of 0.003 inches when tapered membrane removing forceps tip 100 comprises an open tapered membrane removing forceps 300.

In one or more embodiments, first superior face distal end 131 may be separated from second superior face distal end 131 by a distance of less than 0.002 inches or greater than 0.004 inches when tapered membrane removing forceps tip 100 comprises an open tapered membrane removing forceps 300.

FIG. 3B illustrates an isometric view of a partially closed tapered membrane removing forceps 310. In one or more embodiments, a compression of actuation structure 210 may be configured to extend hypodermic tube 250 relative to blank 150. Illustratively, a compression of actuation structure 210 may be configured to gradually close a tapered membrane removing forceps tip 100 from an open tapered membrane removing forceps 300 to a partially closed tapered membrane removing forceps 310. In one or more embodiments, a compression of actuation structure 210 may be configured to decrease a distance between first tapered forceps jaw 110 and second tapered forceps jaw 110. Illustratively, a compression of actuation structure 210 may be configured to decrease a distance between first superior face distal end 131 and second superior face distal end 131.

FIG. 3C illustrates an isometric view of a fully closed tapered membrane removing forceps 320. In one or more embodiments, a compression of actuation structure 210 may be configured to extend hypodermic tube 250 relative to blank 150. Illustratively, a compression of actuation structure 210 may be configured to gradually close a tapered membrane removing forceps tip 100 from a partially closed tapered membrane removing forceps 310 to a fully closed tapered membrane removing forceps 320. In one or more embodiments, first superior face distal end 131 and second superior face distal end 131 may be in contact when a tapered membrane removing forceps tip 100 comprises a fully closed tapered membrane removing forceps 320.

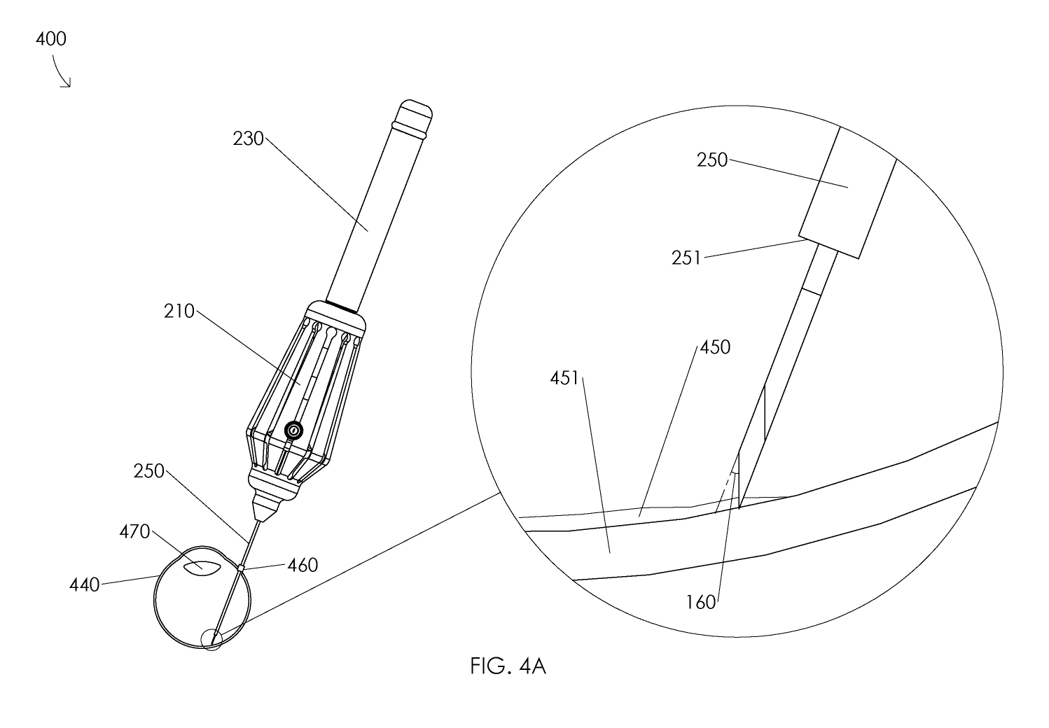

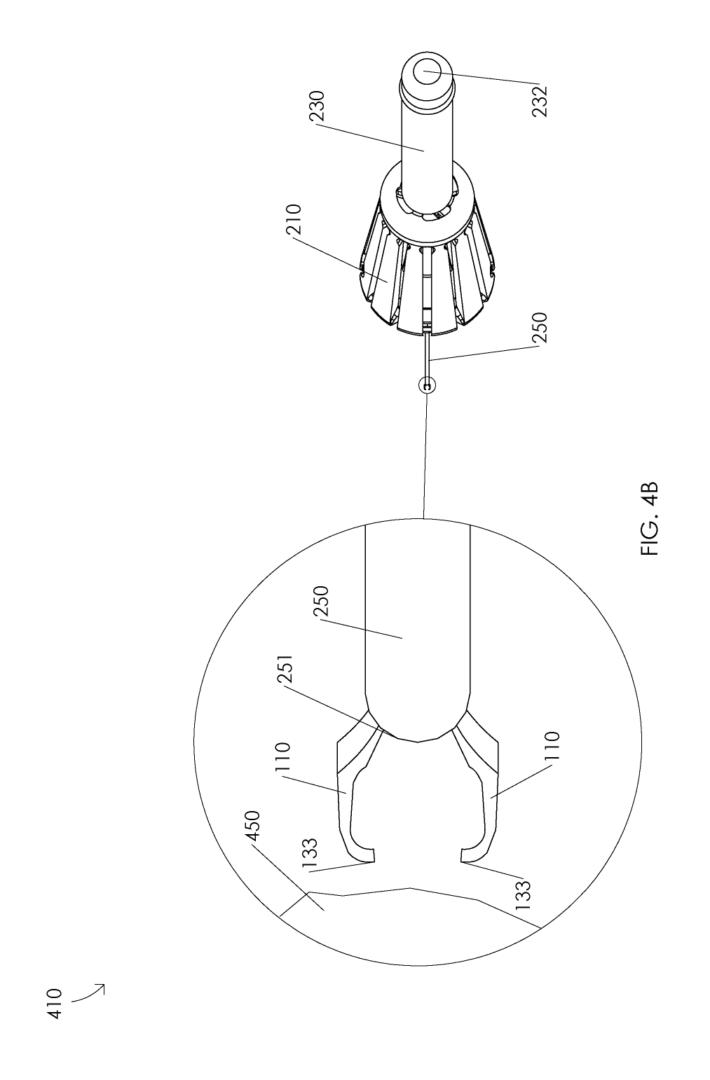

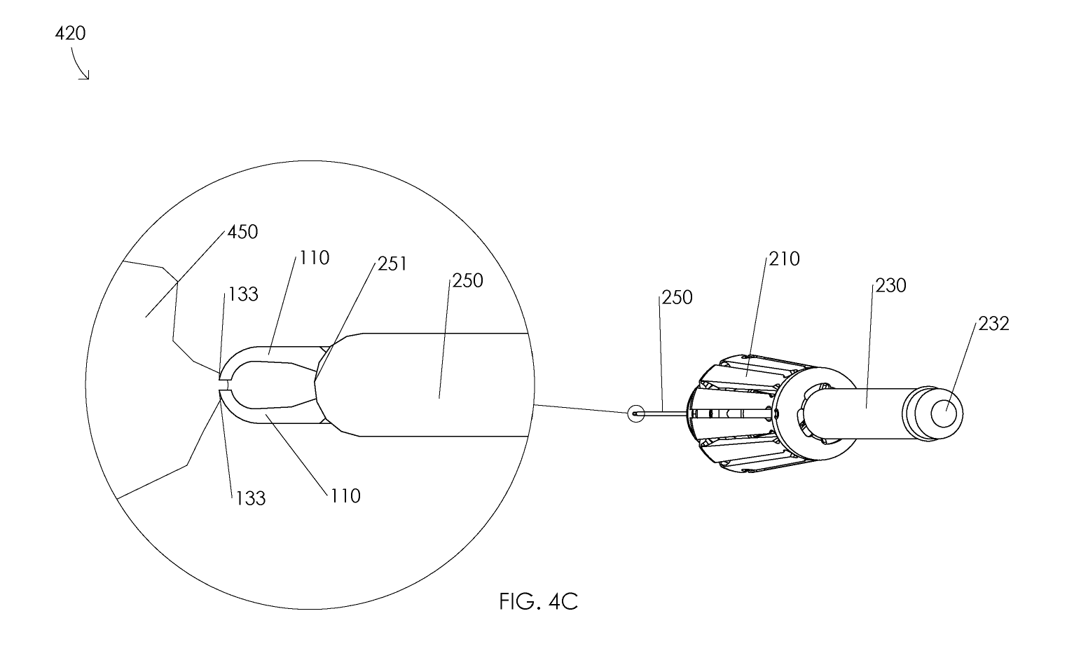

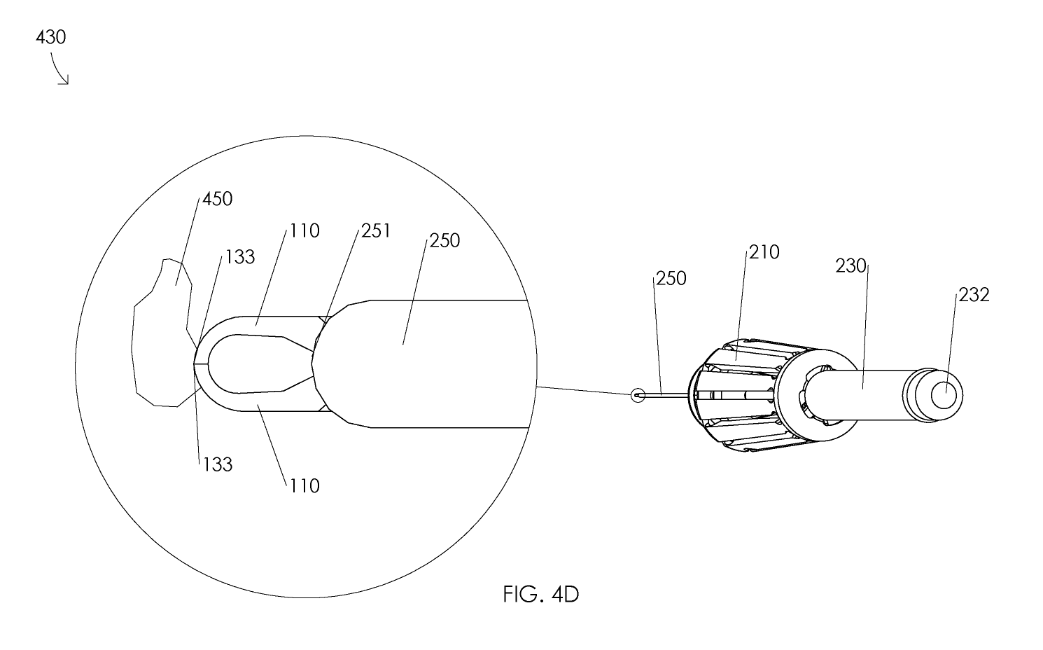

FIGS. 4A, 4B, 4C, and 4D are schematic diagrams illustrating a surgical procedure. FIG. 4A illustrates a posterior segment approach 400. Illustratively, a posterior segment approach 400 may be achieved through a cannula 460 disposed in an incision in a pars plana of an eye 440. In one or more embodiments, a surgeon may begin a posterior segment approach 400 by inserting tapered membrane removing forceps tip 100 and hypodermic tube 250 into cannula 460 and advancing tapered membrane removing forceps tip 100 into an inner portion of eye 440 until superior face distal ends 131 approach a retina 451. Illustratively, a surgeon may be required to perform a posterior segment approach 400 at an angle relative to a sagittal plane of eye 440 to avoid contacting lens capsule 470. In one or more embodiments, a surgeon may be required to perform a posterior segment approach 400 at an angle in a range of 19.0 to 23.0 degrees relative to a sagittal plane of eye 440 to avoid contacting lens capsule 470, e.g., a surgeon may be required to perform a posterior segment approach 400 at an angle of 21.0 degrees relative to a sagittal plane of eye 440 to avoid contacting lens capsule 470. Illustratively, a surgeon may be required to perform a posterior segment approach 400 at an angle of less than 19.0 degrees or greater than 23.0 degrees relative to a sagittal plane of eye 440 to avoid contacting lens capsule 470.

In one or more embodiments, membrane 450 may be disposed over a portion of retina 451. Illustratively, membrane 450 may comprise an internal limiting membrane. In one or more embodiments, membrane 450 may comprise an epiretinal membrane. Illustratively, a surgeon may be required to approach membrane 450 at an angle relative to a line normal to a surface of membrane 450 to avoid contacting lens capsule 470. In one or more embodiments, a surgeon may be required to approach membrane 450 at an angle in a range of 19.0 to 23.0 degrees relative to a line normal to a surface of membrane 450 to avoid contacting lens capsule 470, e.g., a surgeon may be required to approach membrane 450 at an angle of 21.0 degrees relative to a line normal to a surface of membrane 450 to avoid contacting lens capsule 470. Illustratively, a surgeon may be required to approach membrane 450 at an angle of less than 19.0 degrees or greater than 23.0 degrees relative to a line normal to a surface of membrane 450 to avoid contacting lens capsule 470. In one or more embodiments, a surgeon may be required to approach membrane 450 at an angle equal to tapered angle 160 relative to a line normal to a surface of membrane 450 to avoid contacting lens capsule 470. Illustratively, tapered angle 160 may be configured to match a surgeon's required approach angle relative to a line normal to a surface of membrane 450 to avoid contacting lens capsule 470.

FIG. 4B illustrates a membrane approach 410. Illustratively, as a surgeon guides tapered membrane removing forceps tip 100 towards membrane 450 at an angle equal to tapered angle 160 relative to a line normal to a surface of membrane 450, first superior face 130 and second superior face 130 are not visible in the surgeon's line-of-sight. In one or more embodiments, a surgeon may use first superior face superior end 133 and second superior face superior end 133 as a guide to approximate a location of first superior face distal end 131 and second superior face distal end 131. Illustratively, superior face superior end 133 may be separated from superior face distal end 131 by a distance in a range of 0.017 to 0.032 inches, e.g., superior face superior end 133 may be separated from superior face distal end 131 by a distance of 0.021 inches. In one or more embodiments, superior face superior end 133 may be separated from superior face distal end 131 by a distance of less than 0.017 inches or greater than 0.032 inches. In one or more embodiments, guiding tapered membrane removing forceps tip 100 towards membrane 450 at an angle equal to tapered angle 160 relative to a line normal to a surface of membrane 450 may be configured to increase a surgeon's visualization of membrane 450. For example, a surgeon may perform a membrane approach 410 with a maculorhexis forceps tip and visualize a first amount of membrane 450. Illustratively, a surgeon may perform a membrane approach 410 with a tapered membrane removing forceps tip 100 and visualize a second amount of membrane 450. In one or more embodiments, the second amount of membrane 450 may be greater than the first amount of membrane 450. Illustratively, guiding tapered membrane removing forceps tip 100 towards membrane 450 at an angle equal to tapered angle 160 relative to a line normal to a surface of membrane 450 may be configured to increase a surgeon's visualization of retina 451. In one or more embodiments, a surgeon may perform a membrane approach 410 with a maculorhexis forceps tip and visualize a first amount of retina 451. Illustratively, a surgeon may perform a membrane approach 410 with a tapered membrane removing forceps tip 100 and visualize a second amount of retina 451. In one or more embodiments, the second amount of retina 451 may be greater than the first amount of retina 451. Illustratively, tapered angle 160 may be configured to indicate whether a surgeon may be performing a membrane approach 410 at an angle relative to a line normal to a surface of membrane 450 that is configured to avoid contacting lens capsule 470. In one or more embodiments, if first superior face distal end 131 or second superior face distal end 131 is visible in a surgeon's line-of-sight in an orientation wherein first superior face distal end 131 or second superior face distal end 131 is disposed superior relative to first superior face superior end 133 or second superior face superior end 133, then tapered angle 160 may be configured to indicate that the surgeon's approach angle relative to the line normal to a surface of membrane 450 may be increased to avoid contacting lens capsule 470. Illustratively, if first superior face distal end 131 or second superior face distal end 131 is visible in a surgeon's line-of-sight in an orientation wherein first superior face distal end 131 or second superior face distal end 131 is disposed inferior relative to first superior face superior end 133 or second superior face superior end 133, then tapered angle 160 may be configured to indicate that the surgeon's approach angle relative to the line normal to a surface of membrane 450 may be decreased without increasing a risk of contacting lens capsule 470 during a membrane approach 410.

FIG. 4C illustrates a membrane grasping 420. Illustratively, a surgeon may perform a membrane grasping 420 by disposing first superior face distal end 131 and second superior face distal end 131 over membrane 450 and compressing actuation structure 210. In one or more embodiments, a compression of actuation structure 210 may be configured to extend hypodermic tube 250 relative to blank 150, e.g., a compression of actuation structure 210 may be configured to extend hypodermic tube distal end 251 over first tapered forceps jaw 110 and second tapered forceps jaw 110. Illustratively, a compression of actuation structure 210 may be configured decrease a distance between first superior face distal end 131 and second superior face distal end 131 until first superior face distal end 131 and second superior face distal end 131 are only separated by a portion of membrane 450 disposed between first superior face distal end 131 and second superior face distal end 131.

FIG. 4D illustrates a membrane removal 430. Illustratively, after performing a membrane grasping 420, a surgeon may perform a membrane removal 430 by peeling membrane 450 off of a surface of retina 451. In one or more embodiments, a surgeon may maintain a compression of actuation structure 210 to maintain a membrane grasping 420 after a membrane removal 430. Illustratively, after performing a membrane removal 430, a surgeon may withdraw tapered membrane removing forceps tip 100 and hypodermic tube 250 out from cannula 460. In one or more embodiments, a surgeon may release membrane 450 by decompressing actuation structure 210.

The foregoing description has been directed to particular embodiments of this invention. It will be apparent; however, that other variations and modifications may be made to the described embodiments, with the attainment of some or all of their advantages. Specifically, it should be noted that the principles of the present invention may be implemented in any system. Furthermore, while this description has been written in terms of a membrane removing forceps, the teachings of the present invention are equally suitable to any systems where the functionality may be employed. Therefore, it is the object of the appended claims to cover all such variations and modifications as come within the true spirit and scope of the invention.

What is claimed is:

1. An instrument comprising:
a first tapered forceps jaw having a first tapered forceps jaw distal end, a first tapered forceps jaw proximal end, and a first tapered forceps jaw superior surface;
a first superior face of the first tapered forceps jaw having a first superior face distal end and a first superior face proximal end wherein the first superior face distal end is the first tapered forceps jaw distal end;
a first tapered angle between a line tangent to the first tapered forceps jaw superior surface and the first superior face, the first tapered angle in a range of 19.0 to 23.0 degrees;
a second tapered forceps jaw having a second tapered forceps jaw distal end, a second tapered forceps jaw proximal end, and a second tapered forceps jaw superior surface;
a second superior face of the second tapered forceps jaw having a second superior face distal end and a second superior face proximal end wherein the second superior face distal end is the second tapered forceps jaw distal end;
a second tapered angle between a line tangent to the second tapered forceps jaw superior surface and the second superior face, the second tapered angle in a range of 19.0 to 23.0 degrees;
a tapered aperture having a tapered aperture tapered portion, the tapered aperture disposed between a portion of the first tapered forceps jaw and a portion of the second tapered forceps jaw;
a hypodermic tube having a hypodermic tube distal end and a hypodermic tube proximal end wherein the first tapered forceps jaw and the second tapered forceps jaw are at least partially disposed in the hypodermic tube wherein the first tapered forceps jaw distal end and the second tapered forceps jaw distal end extend from the hypodermic tube distal end; and
an actuation structure having an actuation structure distal end and an actuation structure proximal end wherein a compression of the actuation structure is configured to extend the hypodermic tube relative to the first tapered forceps jaw and the second tapered forceps jaw.

2. The instrument of claim 1 further comprising:
a superior face superior end of the first superior face;
a superior face lateral interface of the first superior face; and
a superior face superior interface of the first superior face, the superior face superior interface disposed between the superior face superior end and the first superior face proximal end.

3. The instrument of claim 2 further comprising:
a superior distal shoulder of the first tapered forceps jaw, the superior distal shoulder disposed between the superior face superior end and the superior face lateral interface.

4. The instrument of claim 2 further comprising:
a superior proximal shoulder of the first tapered forceps jaw, the superior proximal shoulder disposed between the superior face lateral interface and the first tapered forceps jaw proximal end.

5. The instrument of claim 2 further comprising:
a lateral projection of the first tapered forceps jaw, the superior proximal shoulder disposed between the superior face lateral interface and the first tapered forceps jaw proximal end.

6. The instrument of claim 1 further comprising:
an inferior face of the first tapered forceps jaw having an inferior face distal end and an inferior face proximal end.

7. The instrument of claim 1 further comprising:
an inferior proximal shoulder of the first tapered forceps jaw.

8. The instrument of claim 1 further comprising:
a removable handle having a removable handle distal end and a removable handle proximal end wherein a portion of the removable handle is disposed in an inner chamber proximal taper of the actuation structure.

9. The instrument of claim 8 further comprising:
a plurality of fingers of the actuation structure, the plurality of fingers disposed in the inner chamber proximal taper of the actuation structure.

10. The instrument of claim 9 further comprising:
a barb head of the removable handle;
a barb base of the removable handle;
a barb channel of the removable handle, the barb channel disposed between the barb head and the barb base; and
an actuation structure interface of the removable handle.

11. The instrument of claim 10 wherein at least a portion of each finger of the plurality of fingers is disposed in the barb channel.

12. The instrument of claim 11 wherein the plurality of fingers are configured to temporarily fix the portion of the removable handle in the inner chamber proximal taper.

13. The instrument of claim 12 wherein the plurality of fingers are configured to temporarily fix the portion of the removable handle in the inner chamber proximal taper by a torsional snap fit.

14. The instrument of claim 13 further comprising:
a setscrew housing of the actuation structure; and
a blank having a blank distal end and a blank proximal end, a portion of the blank disposed in the setscrew housing.

15. The instrument of claim 14 further comprising:
a superior setscrew disposed in the setscrew housing; and
an inferior setscrew disposed in the setscrew housing.

16. The instrument of claim 15 wherein the portion of the blank is disposed between the superior setscrew and the inferior setscrew.

17. The instrument of claim 16 further comprising:
an inner chamber distal taper of the actuation structure.

18. The instrument of claim 17 further comprising:
an inner chamber of the actuation structure, the inner chamber disposed between the inner chamber distal taper and the inner channel proximal taper.

19. The instrument of claim 18 further comprising:
an inner nosecone of the actuation structure.

20. The instrument of claim 19 further comprising:
an inner bore of the removable handle.
